(19) United States
(12) Patent Application Publication
(10) Pub. No.: US 2015/0196477 A1
(43) Pub. Date: Jul. 16, 2015

(54) METHODS OF REDUCING MALODOR AND BACTERIA

(71) Applicant: The Procter & Gamble Company, Cincinnati, OH (US)

(72) Inventors: Cynthia Marie STARK, Fairfield, OH (US); Anthony Charles LANZALACO, Fairfield, OH (US); Andrea Kay HEAPE, Blue Ash, OH (US); Chanchal Kumar GHOSH, West Chester, OH (US)

(21) Appl. No.: 14/598,051

(22) Filed: Jan. 15, 2015

Related U.S. Application Data
(60) Provisional application No. 61/927,814, filed on Jan. 15, 2014.

(51) Int. Cl. A61K 8/81; A61K 8/27; A61Q 15/00; A61K 8/49

(52) U.S. Cl. CPC A61K 8/817; A61K 8/4933; A61K 8/27; A61Q 15/00

(57) ABSTRACT

Methods for reducing bacteria include applying a rinse-off personal care composition including a malodor control polymer to at least a portion of the body of a user. Methods for reducing malodor are also provided, as well as rinse-off personal care compositions used in such methods.

### TECHNICAL FIELD

The present disclosure generally relates to methods of reducing malodor and bacteria on a portion of the body of a user by applying a rinse-off personal care composition comprising a malodor control polymer including a polyvinylamine polymer. Further, the present disclosure generally relates to synergistic benefits of applying rinse-off personal care composition comprising a malodor control polymer with a particulate antimicrobial agent.

### BACKGROUND

It is well established that human malodors are caused by microbial interactions with apocrine gland secretions. Historically, people have attempted to reduce these odors through cleansing and the topical application of deodorant or antiperspirant products. However, it has been observed that even the combination of potent antimicrobials, strong masking perfumes, and rigorous cleansing may not be sufficient to eliminate malodor. Accordingly, it is desirable to provide improved methods for reducing malodor by targeting the microorganisms that create it, including those odor-causing bacteria that reside in human hair follicles.

### SUMMARY

A method of reducing underarm malodor, comprising: applying a rinse-off personal care composition to an underarm of a user, wherein the composition comprises an antimicrobial polyvinylamine copolymer comprising about 95% mol vinyl monomer and about 5% mol vinylformamide monomer.

A method of reducing bacteria on skin, comprising: applying a rinse-off personal care composition to at least a portion of the skin of a user, wherein the composition comprises a malodor control polymer comprising a polyvinylamine copolymer comprising a vinyl monomer and a vinyl formamide monomer.

In accordance with still another example, a method of reducing bacteria comprises applying a rinse-off personal care composition to at least a portion of one of hair follicles or skin. The rinse-off personal care composition comprises from about 0.01% to about 20%, by weight of the composition, of a malodor control polymer comprising a polyvinylamine polymer; from about 0.01% to about 4%, by weight of the composition, of zinc pyrithione; and from about 0.1% to about 10%, by weight of the composition, of zinc carbonate.

In accordance with still another example, a rinse-off personal care composition comprises from about 0.5% to about 5%, by weight of the composition, of a malodor control polymer comprising a polyvinylamine polymer; from about 0.1% to about 0.5%, by weight of the composition, of zinc pyrithione; and from about 0.5% to about 2%, by weight of the composition, of zinc carbonate.

### BRIEF DESCRIPTION OF THE DRAWINGS

FIG. 1 is a graph showing 12 hour self-assessed odor of a placebo composition versus an inventive rinse-off personal care composition comprising a malodor control polymer;

FIG. 2 is a graph showing 24 hour self-assessed odor of a placebo composition versus an inventive rinse-off personal care composition comprising a malodor control polymer;

FIG. 3 is a graph showing the antimicrobial activity in hair follicles of an inventive rinse-off personal care composition comprising a malodor control polymer versus a control composition (no malodor control polymer) at baseline and post treatment;

FIG. 4 is graph showing the antimicrobial activity of an inventive rinse-off personal care composition comprising zinc pyrithione, zinc carbonate, and a malodor control polymer versus a control composition (no malodor control polymer) at baseline and post treatment;

FIG. 5 is a graph showing 12 hour self-assessed odor of a of an inventive rinse-off personal care composition comprising zinc pyrithione, zinc carbonate, and a malodor control polymer versus a control composition (no malodor control polymer); and

FIG. 6 is a graph showing 24 hour self-assessed odor of a of an inventive rinse-off personal care composition comprising zinc pyrithione, zinc carbonate, and a malodor control polymer versus a control composition (no malodor control polymer).

### DETAILED DESCRIPTION

### I. Definitions

As used herein, the following terms shall have the meaning specified thereafter:

“Anhydrous” refers to those compositions, and components thereof, which are substantially free of water.

“Bar soap” refers to compositions intended for topical application to a surface such as skin or hair to remove, for example, dirt, oil, and the like. The bar soaps can be rinse-off formulations, in which the product is applied topically to the skin or hair and then subsequently rinsed within minutes from the skin or hair with water. The product could also be wiped off using a substrate. Bar soaps can be in the form of a solid (e.g., non-flowing) bar soap intended for topical application to skin. The bar soap can also be in the form of a soft solid which is compliant to the body. The bar soap additionally can be wrapped in a substrate which remains on the bar during use.

“Leave-on composition” refers to a composition that is placed on its intended target, like the skin, and is left in place for an extended period of time, generally hours, in order to provide its benefit, like a stick antiperspirant.

“Personal care composition” refers to compositions intended for topical application to skin or hair. The personal care compositions can be, for example, in the form of a liquid, semi-liquid cream, lotion, gel, or solid and are intended for topical application to the skin and/or hair. Examples of personal care compositions can include but are not limited to bar soaps, shampoos, conditioning shampoos, body washes, moisturizing body washes, shower gels, skin cleansers, cleansing milks, in shower body moisturizers, pet shampoos, shaving preparations, etc.

“Rinse-off” means the intended product usage includes application to skin and/or hair followed by rinsing and/or wiping the product from the skin and/or hair within a few seconds to minutes of the application step.

“STnS” refers to sodium trideceth(n) sulfate, wherein n can define the average number of moles of ethoxylate per molecule.

“Structured” refers to having a rheology that can confer stability on the personal care composition. A cleansing phase can be considered to be structured if the cleansing phase has one or more following characteristics: (a) Zero Shear Viscosity of at least 100 Pascal-seconds (Pa-s), at least about 200 Pa-s, at least about 500 Pa-s, at least about 1,000 Pa-s, at least about 1,500 Pa-s, or at least about 2,000 Pa-s; (b) A Structured Domain Volume Ratio as measured by the Ultracentrifugation Method described hereinafter, of greater than about 40%, at least about 45%, at least about 50%, at least about 55%, at least about 60%, at least about 65%, at least about 70%, at least about 75%, at least about 80%, at least about 85%, or at least about 90%; or (c) A Young's Modulus of greater than about 2 Pascals (Pa), greater than about 10 Pa, greater than about 20 Pa, greater than about 30 Pa, greater than about 40 Pa, greater than about 50 Pa, greater than about 75 Pa, or greater than about 100 Pa.

“Substantially free of” refers to about 2% or less, about 1% or less, or about 0.1% or less of a stated ingredient. “Free of” refers to no detectable amount of the stated ingredient or thing.

### II. Rinse-off Personal Care Compositions

As noted above, several technical approaches can be used to reduce body odor after the odor-causing material has formed, like the use of increased perfume levels and odor neutralizers. Another approach can be to target the organisms, like bacteria, that contribute to body odor by using an antimicrobial agent. Some examples of bacteria on the skin that can cause body odor can include S. epidermidis and C. mucifaciens. A genuine malodor reduction can provide a sensory (e.g., self-assessed body odor) malodor reduction and/or analytically measurable (e.g., hair pluck method) bacteria control. Thus, if a composition can deliver a genuine malodor reduction, the composition can reduce or prevent the formation of at least some of the malodors on the body.

In addition, the hair follicles and skin crevices can play an important role in odor control as they house odor producing bacteria. Current rinse-off formulations, particularly body washes and bar soaps, can be ineffective in targeting odor-producing bacteria in the hair follicle and skin crevices. During typical use, such formulations can be applied to an underarm and then rinsed away, only targeting bacteria on the surface of the skin. This can result in a transient reduction in underarm odor as antimicrobial agents can be washed away during rinsing.

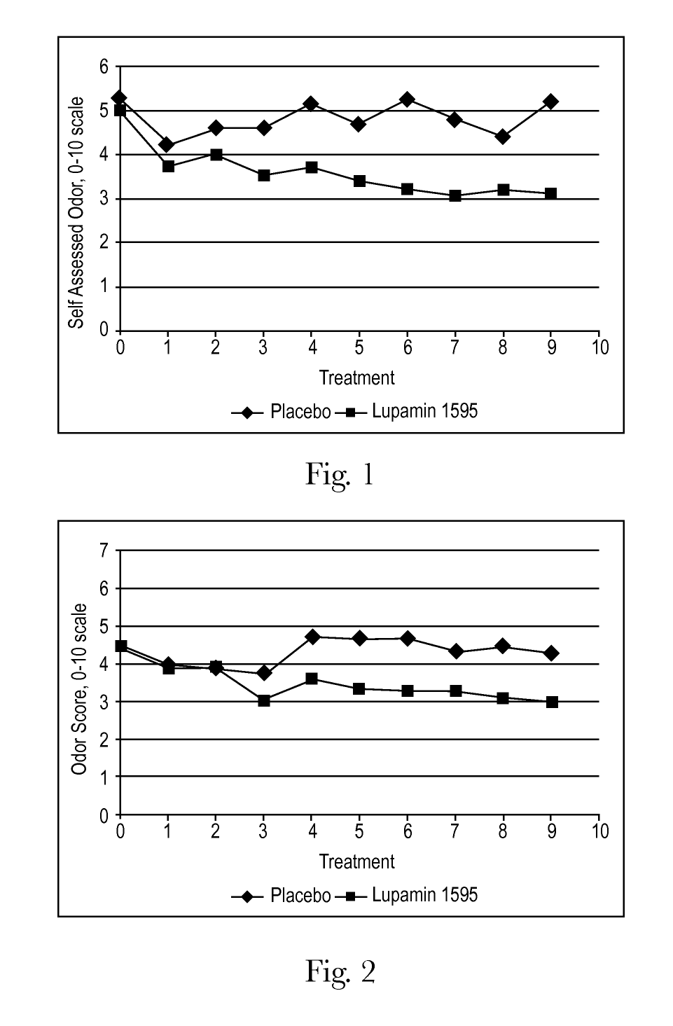

While polyvinylamine polymers (PVam) can sometimes be used in leave-on products as odor neutralizers via odor absorption or antimicrobials in hair follicles, the present inventors have surprisingly discovered that PVam can be used in rinse-off personal care compositions to effect a reduction of malodor through antimicrobial action on the skin. Because PVam are water soluble, delivering such actives in a rinse-off personal care composition with limited residence time on the skin would not be expected to provide adequate deposition on skin to affect noticeable odor control. Nonetheless, compositions containing PVam (e.g., Lupamin® 1595) showed significant body odor reduction efficacy in self-assessed odor clinical trials. This is illustrated in FIGS. 1 and 2 where subjects performed self-body odor assessments by sniffing under each underarm twice daily at 12 and 24 hours post-product application and record the level of odor on a scale from 0-10. The composition containing PVam (Lupamin® 1595) had superior anti-odor efficacy at both 12 hours (FIG. 1) and 24 hours (FIG. 2) following application of the rinse-off composition versus a placebo formulation that did not contain PVam (Lupamin® 1595). Odor differences of up to 2 units were observed.

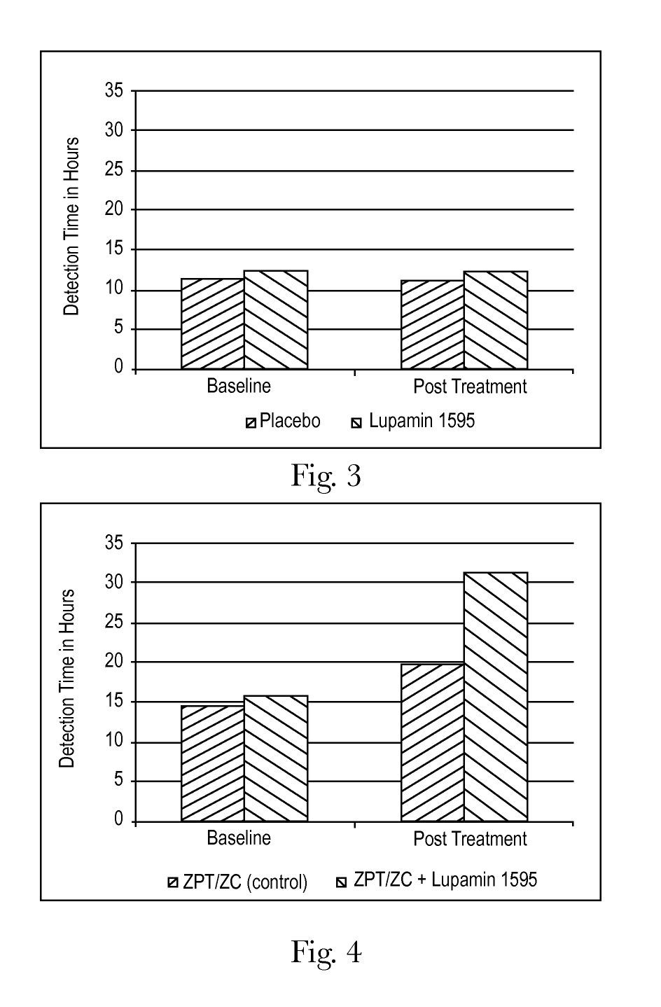

In addition, FIG. 3 supports the rationale that the odor reduction from PVam is occurring on the skin versus in the hair follicle. To look at activity in a hair follicle, hairs are plucked from a site after application of the target composition and then assessed for microbial activity based on detection time. So, an increase in detection time would show less microbial activity in a follicle as it is taking longer for the microbes to grow and, thus, more antimicrobial activity as a result of the target composition. As can be seen from FIG. 3, a rinse-off composition including PVam (Lupamin® 1595) did not exhibit noticeable improvement in antimicrobial activity in the hair follicle when compared to baseline and a placebo formulation. Thus, a reduction in malodor resulting from application of a rinse-off personal care composition including PVam can likely be attributed to PVam controlling odor-causing bacteria on the surface of the skin versus in the follicle.

As noted above, hair follicles and skin crevices can play an important role in odor control. Malodor-causing bacteria can often be present within hair follicles and skin crevices and conventional cleansing products can have difficulty reaching and controlling bacteria in these areas. This can be especially true in the underarm, which includes the presence of three secreting glands—eccrine, sebaceous and apocrine—in an area densely populated with hair follicles and skin crevices. The underarm secreting glands provide the bacteria with water, nutrients, and odor precursor materials. In this environment, bacteria are free to metabolize odor precursor compounds producing malodors that can overwhelm even the strongest masking perfumes.

One way to target bacteria in the hair follicles and skin crevices is the use of particulate antimicrobial actives. However, antimicrobial efficacy alone is not a sufficient prerequisite for treating underarm body odor as the microbial and anatomical environment responsible for the odor is unique to the underarm and requires a specially designed antimicrobial system. In addition, the fact that a given material can successfully treat dandruff is no guarantee that such a material can treat underarm body odor. For example, in one clinical study where subjects washed their underarms each morning for nine consecutive days with a dandruff shampoo containing selenium sulfide and then rinsed off their underarms with water and odor assessments were made at baseline, on day 4, and at the end of the study (day 9) by trained odor judges employing the standard odor grading methodology, the composition containing the antimicrobial agent selenium sulfide did not reduce underarm body odor.

Once an appropriate underarm anti-odor antimicrobial is identified, like particulate zinc pyrithione, then comes the task of depositing it into the underarm hair follicle and/or skin crevices. Cationic polymers have been used to help deposit anti-dandruff actives on the scalp. However, the effectiveness of these types of materials to deposit underarm anti-odor actives into hair follicles can depend on a variety of properties like, for example, solubility, molecular weight, and structure. Cationic polymer deposition aids that either bind the antimicrobial agent too tightly and/or “bury” the antimicrobial agent in the polymer on the skin will reduce the exposure of the odor causing bacteria to the antimicrobial agent potentially rendering the agent biologically inactive. Thus, you could get a lot of deposition but not a lot of efficacy. Net, in controlling underarm body odor, the effectiveness of enhanced deposition of the antimicrobial agent onto skin will be dependent on the specific properties of the antimicrobial agent and polymer and not simply on how much of the antimicrobial agent is deposited. The Hair Pluck method described in this application is a highly effective way to determine antimicrobial bioactivity at the anatomical location responsible for odor production.

It has surprisingly been discovered that PVam can provide a delivery and/or retention benefit for antimicrobial agents, like ZPT. In fact, it has a synergistic effect when used in a rinse-off personal care composition including a combination of zinc pyrithione (ZPT) and zinc carbonate (ZC). This combination exhibited a strong antimicrobial effect against key odor-causing bacteria in the hair follicles. This can be seen in FIG. 4, where the rinse-off personal care composition including PVam (Lupamin® 1595) and a combination of ZPT and ZC exhibited superior antimicrobial activity when compared to a control composition without PVam. Thus, inclusion of PVam can provide a delivery and/or retention benefit for the ZPT/ZC, and the rinse-off personal care composition including PVam (Lupamin® 1595), ZPT, and ZC had an unexpectedly, stronger antimicrobial effect in the hair follicle.

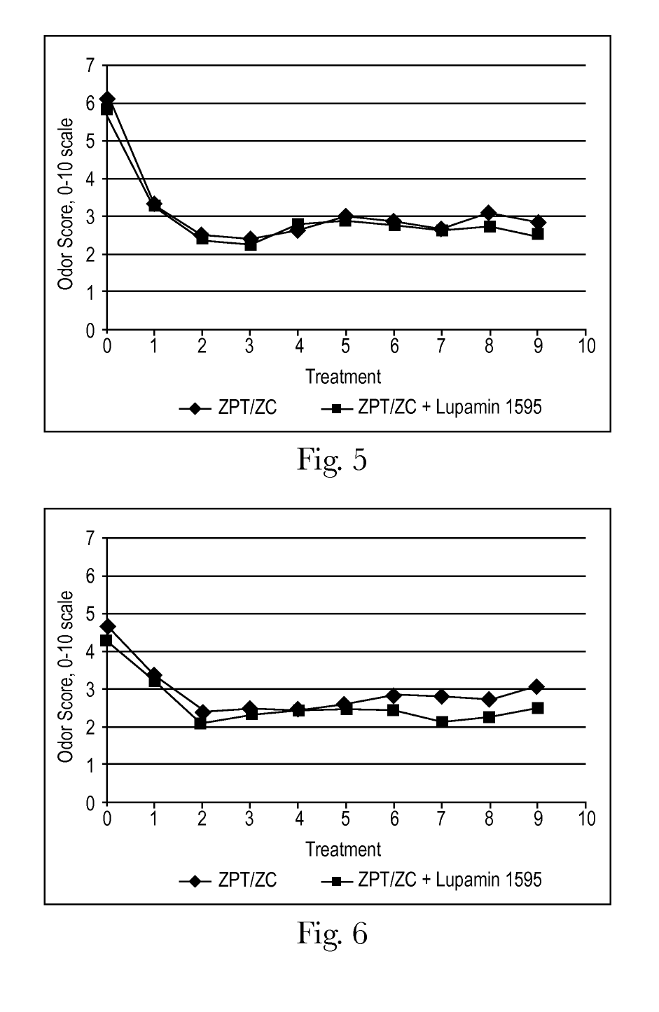

The same phenomenon was also seen in self-assessed odor control where the PVam/ZPT/ZC containing composition provided a slight advantage in self-assessed odor control at 12 hours (FIG. 5), but at 24 hours following application (FIG. 6), a more noticeable self-assessed odor control was exhibited. This long-lasting odor control can further reveal a delivery and/or retention benefit for ZPT/ZC by inclusion of PVam in a rinse-off personal care composition. Accordingly, this benefit can allow particulate antimicrobial agents to be effectively deposited within skin crevices and/or hair follicles upon application of the rinse-off personal care composition as described herein, such that during a wash/rinse period, at least a portion of these antimicrobial agents can be retained to provide an unexpected antimicrobial effect as well as longer-lasting odor control. Without being limited by theory, it is believed that the lower molecular weight and linear structure of the PVam tested offer the best opportunities for follicular penetration without the negative consequences of “deactivating” the antimicrobial agent by “burying” it within a polymer or through strong polymer/particulate binding.

Rinse-off personal care compositions may come in many forms. For example, a personal care composition may be in a liquid form and could be a body wash, shampoo, conditioning shampoos, moisturizing body washes, shower gels, skin cleansers, cleansing milks, in shower body moisturizers, pet shampoos, shaving preparations, etc. Rinse-off personal care compositions may also be in a solid form, like in a bar soap, which can also be in many shapes and forms (e.g., a rectangle or in a powder or pellet form). Examples of some suitable rinse-off personal care compositions are described in U.S. Patent Application Publication No. 2013/0045907.

A. Malodor Control Polymers

Rinse-off personal care compositions as described herein can include a malodor control polymer. Malodor control polymers can be water soluble and can be formed from a polyvinylamine polymer having a primary amine group. Such malodor control polymers are referred to as PVam. A rinse-off personal care composition can include from about 0.01% to about 20%, by weight of the composition, of a malodor control polymer. In certain examples, the rinse-off personal care composition may also comprise from about 0.05%, about 0.1%, about 0.5%, about 1.0%, about 1.5%, about 2%, about 3%, about 5%, to about 2.0%, about 3.0%, about 5%, about 10%, about 15%, about 20%, or any combination thereof, by weight of the composition, of the malodor control polymer. While these malodor control polymers can often come in solution form, the amounts of polymer, can be by active percentage, not percentage of raw material.

A PVam can be a linear polymer with pendent, primary amine groups directly linked to the main chain of alternating carbons. PVams can be manufactured from hydrolysis of poly(N-vinylformamide) (PVNF) which can result in the conversion of formamide units to amino groups as described by the following formula (Ila):

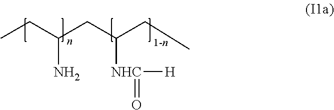

where n is a number from 0.1 to 0.99 depending on the degree of hydrolysis. For instance, in 95% hydrolyzed PVam polymer, n will be 0.95 while 5% of the polymer will have formamide units.

PVams can be partially hydrolyzed meaning that 1% to 99%, 30% to 99%, 50% to 99%, 70% to 99%, 80% to 99%, 85% to 99%, 90% to 99%, 95% to 99%, 97% to 99%, or about 99% of the PVam can be hydrolyzed. It has been found that a high degree of hydrolysis of PVam can increase the resulting polymer's ability to mitigate the odors.

PVams that can be hydrolyzed may have an average molecular weight (MW) of 5,000 to 350,000. Suitable hydrolyzed PVams can be commercially available from BASF. Some examples include Lupamin® 9095, 9030, 9010, 5095, and 1595.

B. Antimicrobial Agents

As noted herein, rinse-off personal care compositions can further include antimicrobial agents. Exemplary rinse-off personal care compositions can employ a particulate antimicrobial agent. The antimicrobial agent and carrier material (and/or other solvent-acting material ingredients) can be chosen such that the antimicrobial agent remains as a solid particulate within the final formulation and upon application to the skin; that is, the antimicrobial agent is not completely solubilized prior to use. Remaining in particulate form within the final formulation and upon application to the skin enables at least a portion of the antimicrobial agent to deposit into skin crevices and/or hair follicles, and to survive rinsing. Microscopy can enable one to determine the presence of discrete antimicrobial agent particles within the final formulation. The antimicrobial agent can range from completely insoluble in the personal care composition; to substantially insoluble in the composition; to less than 5% soluble; to less than 1% soluble, by weight of the antimicrobial agent.

Particulate Antimicrobial agents can generally range in particle size from about 0.1 μm to about 100 μm and can be even smaller like, from about 0.2 μm to about 50 μm; from about 0.5 μm to about 20 μm; or from about 1 μm to about 10 μm. It should be appreciated that not all of the particulate antimicrobial agent within a given product necessarily falls within the above range and that the particle distribution may be normal or not. It is believed that antimicrobial particles in the 0.1 μm to 10 μm size range can deposit into hair follicles, while larger particles (e.g., 10-100 μm or even larger) may deposit into skin folds, wrinkles, crevices or other surface irregularities that can be present.

Particle size can be measured using light scattering methods. In one example, particle size can be determined with a Horiba LA-950 Laser Diffraction Particle Size Analyzer. This instrument uses the principal of low-angle Fraunhofer Diffraction and Light Scattering from the particles as the means for particle size determination.

Staphylococcus epidermidis and corynebacterium mucifaciens are two key odor-causing bacteria, and generally are associated with the human underarm. Rinse-off personal care compositions designed for managing underarm malodor can include antimicrobial agents having a

Minimal Inhibitory Concentration (“MIC”) of less than or equal to 2,500 μg/ml, 1,000 μg/ml, 500 μg/ml, or 100 μg/ml against at least one strain of at least one of these bacteria. MIC values can be obtained using a traditional broth dilution microbiological technique, such as that described in the following journal article: Andrews, J. M., “Determination of minimum inhibitory concentrations”, Journal of Antimicrobial Chemotherapy 48 (supl. 1): 5-16, 2001. Suitable antimicrobial agents can include, but are not limited to, metals (e.g., Zn, Cu, Al, Ti, Sn, Bi, and Ag), metal salts (e.g., zinc carbonate, copper sulfate, and zinc gluconate), metal pyrithione salts (e.g., ZPT and CuPT), zeolites, metal zeolites, quaternary ammonium (quat) compounds (e.g., cetyl pyridinium chloride, and benzylalkonium chloride), quat bound clays, metal bound clays, and PolyAspirin (e.g., as described in PCT publication no. WO 2008/034019). An antimicrobial agent can be employed in the rinse-off personal care compositions at levels of from about 0.01% to about 10%; other levels may however also be possible. In certain examples, the rinse-off personal care composition may also comprise from about 0.05%, about 0.1%, about 0.2%, about 0.3%, about 0.4%, about 0.5%, about 1.0%, to about 0.5%, about 1.0%, about 2.0%, about 3.0%, about 4%, about 5%, about 10%, or any combination thereof, by weight of the composition, of the antimicrobial agent. Due to differences in the antimicrobial potency of the various particulate antimicrobial agents formulation levels may need to be adjusted accordingly. For example, compositions containing zinc carbonate alone may need to be formulated at the upper range of the listed concentration (2-10%) to achieve optimum odor and follicular bacterial control.

A rinse-off personal care composition can contain a zinc-containing antimicrobial agent.

Such agents can include, for example, a zinc salt of 1-hydroxy-2-pyridinethione (known as “zinc pyrithione” or “ZPT”), for example, a mercaptopyridine-N-oxide zinc salt. The ZPT can be made by reacting 1-hydroxy-2-pyridinethione (i.e., pyrithione acid) or a soluble salt thereof with a zinc salt (e.g. zinc sulfate) to form a zinc pyrithione precipitate as illustrated in U.S. Pat. No. 2,809,971 and the zinc pyrithione can be formed or processed into platelet ZPT using, for example, sonic energy as illustrated in U.S. Pat. No. 6,682,724.

Zinc pyrithione can take the form of particulates, platelets, or a combination thereof. Particulate ZPT can, for example, have an average particle size from about 0.1 μm to about 20 μm or from about 0.2 μm to about 10 μm.

Other suitable examples of zinc salts useful herein can include the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof.

Other non-limiting zinc containing materials can include zinc-containing layer materials (“ZLM's”). ZLM's can typically be those materials with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). ZLM's may have zinc incorporated in the layers and/or be components of the gallery ions. Other suitable ZLMs are described in U.S. Patent Application Publication No. 2008/0138441.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a product or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}.nH₂O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y/n}.nH₂O where the two metal ion may be different; if they are the same and represented by zinc, the formula simplifies to [Zn₁₊ₓ(OH)₂]^{2x+}2xA⁻.nH₂O. This latter formula represents (where x=0.4) common materials such as zinc hydroxychloride and zinc hydroxynitrate. These are related to hydrozincite as well wherein a divalent anion replaces the monovalent anion. These materials can also be formed in situ in a product or in or during a production process.

These classes of ZLM's represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

A personal care composition can contain a combination of antimicrobial agents. For example, a combination of ZPT and a ZLM (such as, for example, basic zinc carbonate) can be used. One exemplary combination includes from about 0.025% to about 0.5% particulate zinc pyrithione and from about 0.1% to about 2.0% of zinc carbonate. In certain examples, a rinse-off personal care composition can include from about 0.01% to about 4% of zinc pyrithione and from about 0.1% to about 10% zinc carbonate; and in certain examples, from about 0.1% to about 0.5% of zinc pyrithione and from about 0.5% to about 2% zinc carbonate.

Additionally, a personal care composition can include a combination of ZPT and other zinc salts including, for example, the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof.

The combination of zinc-containing materials can provide enhanced antimicrobial benefits and synergistic effects (e.g., improved antimicrobial efficacy). In fact, using particulate antimicrobial agents having a combination of zinc-containing materials (e.g., zinc pyrithione and zinc carbonate) in a rinse-off personal care composition can further control odor reduction. This synergistic effect can result, for example, in controlling odor for at least 12 hours after application of the rinse-off personal care composition, or even for at least 24 hours after application of the rinse-off personal care composition, or even for at least 48 hours after application of the rinse-off personal care composition.

Many personal care compositions can be water-based (e.g., rinse-off personal care compositions). It should be understood that an amount of water can be lost, i.e. evaporated, during a process of making a personal care composition, or subsequently, with water being absorbed by surrounding packaging (e.g. a cardboard carton), and the like. Thus, a personal care composition can also include materials that tend to bind the water such that the water can be maintained in the personal care composition at the desired levels. Examples of such materials can include carbohydrate structurants and humectants such as glycerin. However, it will be appreciated that a personal care composition can be anhydrous.

C. Other Ingredients

A variety of optional ingredients can also be added to a personal care composition. Such suitable ingredients can also include, but are not limited to, structurants, humectants, fatty acids, inorganic salts, and other antimicrobial agents or actives.

A personal care composition can also optionally include hydrophilic structurants such as carbohydrate structurants and gums. Some suitable carbohydrate structurants can include raw starch (corn, rice, potato, wheat, and the like) and pregelatinized starch. Some suitable gums can include carrageenan and xanthan gum. A personal care composition may include from about 0.1% to about 30%, from about 2% to about 25%, or from about 4% to about 20%, by weight of the personal care composition, of a carbohydrate structurant.

A personal care composition can also optionally include one or more humectants. Examples of such humectants can include polyhydric alcohols. Further, humectants such as glycerin can be included the personal care composition as a result of production or as an additional ingredient. For example, glycerin can be a by-product after saponification of the personal care composition. Including additional humectant can result in a number of benefits such as improvement in hardness of the personal care composition, decreased water activity of the personal care composition, and reduction of a weight loss rate of the personal care composition over time due to water evaporation.

A personal care composition can optionally include inorganic salts. Inorganic salts can help to maintain a particular water content or level of the personal care composition and improve hardness of the personal care composition. The inorganic salts can also help to bind the water in the personal care composition to prevent water loss by evaporation or other means. A personal care composition can optionally include from about 0.01% to about 15%, from about 1% to about 12%, or from about 2.5% to about 10.5%, by weight of the personal care composition, of inorganic salt. Examples of suitable inorganic salts can include magnesium nitrate, trimagnesium phosphate, calcium chloride, sodium carbonate, sodium aluminum sulfate, disodium phosphate, sodium polymetaphosphate, sodium magnesium succinate, sodium tripolyphosphate, aluminum sulfate, aluminum chloride, aluminum chlorohydrate, aluminum-zirconium trichlorohydrate, aluminum-zirconium trichlorohydrate glycine complex, zinc sulfate, ammonium chloride, ammonium phosphate, calcium acetate, calcium nitrate, calcium phosphate, calcium sulfate, ferric sulfate, magnesium chloride, magnesium sulfate, and tetrasodium pyrophosphate.

A personal care composition can optionally further include one or more additional antimicrobial agents that can serve to further enhance antimicrobial effectiveness of the personal care composition. A personal care composition can include, for example, from about 0.001% to about 2%, from about 0.01% to about 1.5%, or from about 0.1% to about 1%, by weight of the personal care composition, of additional antimicrobial agent(s). Examples of suitable antimicrobial agents can include carbanilides, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid, and other organic acids. Other suitable antimicrobial agents are described in U.S. Pat. No. 6,488,943.

D. Liquid Personal Care Compositions

Exemplary liquid rinse-off personal care compositions can include an aqueous carrier, which can be present at a level of from about 5% to about 95%, or from about 60% to about 85%. The aqueous carrier may comprise water, or a miscible mixture of water and organic solvent. Non-aqueous carrier materials may also be employed.

Such rinse-off personal care compositions may include one or more detersive surfactants. The detersive surfactant component can be included to provide cleaning performance to the product. The detersive surfactant component in turn comprises anionic detersive surfactant, zwitterionic or amphoteric detersive surfactant, or a combination thereof. A representative, non-limiting, list of anionic surfactants includes anionic detersive surfactants for use in the compositions can include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In one example, the anionic surfactant can be sodium lauryl sulfate or sodium laureth sulfate. The concentration of the anionic surfactant component in the product can be sufficient to provide a desired cleaning and/or lather performance, and generally ranges from about 2% to about 50%.

Amphoteric detersive surfactants suitable for use in the rinse-off personal care compositions are well known in the art, and can include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoacetate, disodium lauroamphoacetate disodium cocodiamphoacetate, and mixtures thereof. Amphoacetates and diamphoacetates can also be used.

Zwitterionic detersive surfactants suitable for use in the rinse-off personal care compositions are well known in the art, and can include those surfactants broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

The personal care composition can comprise one or more phases. Such personal care compositions can include a cleansing phase and/or a benefit phase (i.e., a single- or multi-phase composition). Each of a cleansing phrase or a benefit phase can include various components. The cleansing phase and the benefit phase can be blended, separate, or a combination thereof. The cleansing phase and the benefit phase can also be patterned (e.g., striped).

The cleansing phase of a personal care composition can include at least one surfactant. The cleansing phase may be an aqueous structured surfactant phase and be present at from about 5% to about 20%, by weight of the personal care composition. Such a structured surfactant phase may include sodium trideceth(n) sulfate, hereinafter STnS, wherein n can define average moles of ethoxylation. n can range, for example, from about 0 to about 3; from about 0.5 to about 2.7, from about 1.1 to about 2.5, from about 1.8 to about 2.2, or n can be about 2. When n can be less than 3, STnS can provide improved stability, improved compatibility of benefit agents within the personal care compositions, and increased mildness of the personal care compositions, such described benefits of STnS are disclosed in U.S. patent application Ser. No. 13/157,665.

The cleansing phase can also comprise at least one of an amphoteric surfactant and a zwitterionic surfactant. Suitable amphoteric or zwitterionic surfactants (in addition to those cited herein) can include, for example, those described in U.S. Pat. No. 5,104,646 and U.S. Pat. No. 5,106,609.

A cleansing phase can comprise a structuring system. A structuring system can comprise, optionally, a non-ionic emulsifier, optionally, from about 0.05% to about 5%, by weight of the personal care composition, of an associative polymer; and an electrolyte.

The personal care composition can be optionally free of sodium lauryl sulfate, hereinafter SLS, and can comprise at least a 70% lamellar structure. However, the cleansing phase could comprise at least one surfactant, wherein the at least one surfactant includes SLS. Suitable examples of SLS are described in U.S. patent application Ser. No. 12/817,786.

As noted herein, rinse-off personal care compositions can also include a benefit phase. The benefit phase can be hydrophobic and/or anhydrous. The benefit phase can also be substantially free of surfactant. A benefit phase can also include a benefit agent. In particular, a benefit phase can comprise from about 0.1% to about 50%, by weight of the personal care composition, of the benefit agent. The benefit phase may comprise less benefit agent, for example, from about 0.5% to about 20%, by weight of the personal care composition, of the benefit agent. Examples of suitable benefit agents can include petrolatum, glyceryl monooleate, mineral oil, natural oils, and mixtures thereof. Additional examples of benefit agents can include water insoluble or hydrophobic benefit agents. Other suitable benefit agents are described in U.S. patent application Ser. No. 13/157,665.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, vegetable oils, sunflower seed oil, soybean oil, vegetable oil derivatives, coconut oil and derivatized coconut oil, cottonseed oil and derivatized cottonseed oil, jojoba oil, cocoa butter, and combinations thereof.

Non-limiting examples of alkyl esters suitable for use as hydrophobic skin benefit agents herein can include isopropyl esters of fatty acids and long chain esters of long chain (i.e. C10-C24) fatty acids, e.g., cetyl ricinoleate, non-limiting examples of which can include isopropyl palmitate, isopropyl myristate, cetyl riconoleate, and stearyl riconoleate. Other example can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of polyglycerin fatty acid esters suitable for use as hydrophobic skin benefit agents herein can include decaglyceryl distearate, decaglyceryl diisostearate, decaglyceryl monomyriate, decaglyceryl monolaurate, hexaglyceryl monooleate, and combinations thereof.

E. Solid Personal Care Compositions

As noted herein, personal care compositions can take on numerous forms. One suitable form is that of a solid personal care composition. Solid compositions can take many forms like powder, pellets, bars, etc. These forms will generally be described herein as bar soap, but it should be understood that the solid composition could be in another form or shape. One example of a bar soap personal care composition can include from about 0.1% to about 35%, by weight of the personal care composition, of water, from about 45% to about 99%, by weight of the personal care composition, of soap, and from about 0.01% to about 5%, by weight of the personal care composition, of a particulate antimicrobial agent. Another suitable antimicrobial bar soap can include, for example, from about 0.1% to about 30%, by weight of the personal care composition, of water, from about 40% to about 99%, by weight of the personal care composition, of soap, and from about 0.25% to about 3%, by weight of the personal care composition, of a particulate antimicrobial agent.

Bar soap compositions can be referred to as conventional solid (i.e. non-flowing) bar soap compositions. Some bar soap composition comprise convention soap, while others contain synthetic surfactants, and still others contain a mix of soap and synthetic surfactant. Bar compositions may include, for example, from about 0% to about 45% of a synthetic anionic surfactant. An example of a suitable conventional soap can include milled toilet bars that are unbuilt (i.e. include about 5% or less of a water-soluble surfactancy builder).

A personal care bar composition can include, for example from about 45% to about 99% or from about 50% to about 75%, by weight of the personal care composition, of soap. Such soaps can include a typical soap, i.e., an alkali metal or alkanol ammonium salt of an alkane- or alkene monocarboxylic acid. Sodium, magnesium, potassium, calcium, mono-, di- and tri-ethanol ammonium cations, or combinations thereof, can be suitable for a personal care composition. The soap included in a personal care composition can include sodium soaps or a combination of sodium soaps with from about 1% to about 25% ammonium, potassium, magnesium, calcium, or a mixture of these soaps. Additionally, the soap can be well-known alkali metal salts of alkanoic or alkenoic acids having from about 12 to about 22 carbon atoms or from about 12 to about 18 carbon atoms. Another suitable soap can be alkali metal carboxylates of alkyl or alkene hydrocarbons having from about 12 to about 22 carbon atoms. Additional suitable soap compositions are described in U.S. patent application Ser. No. 13/036,889.

A personal care composition can also include soaps having a fatty acid. For example, one bar soap composition could use from about 40% to about 95% of soluble alkali metal soap of C₈-C₂₄ or C₁₀-C₂₀ fatty acids. The fatty acid may, for example, have a distribution of coconut oil that can provide a lower end of a broad molecular weight range or a fatty acid distribution of peanut or rapeseed oil, or their hydrogenated derivatives, which can provide an upper end of the broad molecular weight range. Other such compositions can include a fatty acid distribution of tallow and/or vegetable oil. The tallow can include fatty acid mixtures that can typically have an approximate carbon chain length distribution of 2.5% C₁₄, 29% C₁₆, 23% C₁₈, 2% palmitoleic, 41.5% oleic, and 3% linoleic. The tallow can also include other mixtures with a similar distribution, such as fatty acids derived from various animal tallows and/or lard. In one example, the tallow can also be hardened (i.e., hydrogenated) such that some or all unsaturated fatty acid moieties can be converted to saturated fatty acid moieties.

Suitable examples of vegetable oil include palm oil, coconut oil, palm kernel oil, palm oil stearine, soybean oil, and hydrogenated rice bran oil, or mixtures thereof, since such oils can be among more readily available fats. One example of a suitable coconut oil can include a proportion of fatty acids having at least 12 carbon atoms of about 85%. Such a proportion can be greater when mixtures of coconut oil and fats such as tallow, palm oil, or non-tropical nut oils or fats can be used where principle chain lengths can be C₁₆ and higher. The soap included in a personal care composition can be, for example, a sodium soap having a mixture of about 67-68% tallow, about 16-17% coconut oil, about 2% glycerin, and about 14% water.

Soap included in a personal care composition can also be unsaturated in accordance with commercially acceptable standards. For example, a soap included in a personal care composition could include unsaturation in a range of from about 37% to about 45% of saponified material.

Soaps included in a personal care composition can be made, for example, by a classic kettle boiling process or modern continuous soap manufacturing processes wherein natural fats and oils such as tallow or coconut oil or their equivalents can be saponified with an alkali metal hydroxide using procedures well known to those skilled in the art. Soap can also be made by neutralizing fatty acids such as lauric (C₁₂), myristic (C₁₄), palmitic (C₁₆), or stearic (C₁₈) acids, with an alkali metal hydroxide or carbonate.

Soap included in a personal care composition could also be made by a continuous soap manufacturing process. The soap could be processed into soap noodles via a vacuum flash drying process. One example of a suitable soap noodle comprises about 67.2% tallow soap, about 16.8% coconut soap, about 2% glycerin, and about 14% water, by weight of the soap noodle. The soap noodles can then be utilized in a milling process to finalize a personal care composition.

### III. Methods of Use and Methods of Reducing Malodor and Bacteria

Rinse-off personal care compositions can be applied by a variety of means, including by rubbing, wiping or dabbing with hands or fingers, or by means of an implement and/or delivery enhancement device. Non-limiting examples of implements can include a sponge or sponge-tipped applicator, a mesh shower puff, a swab, a brush, a wipe (e.g., wash cloth), a loofah, and combinations thereof. Non-limiting examples of delivery enhancement devices can include mechanical, electrical, ultrasonic and/or other energy devices. Employment of an implement or device may help delivery of the particulate antimicrobial agent to target regions, such as, for example, hair follicles and undulations that can exist in the underarm. A rinse-off care product may be sold together with such an implement or device. Alternatively, an implement or device can be sold separately but contain indicium to indicate usage with a rinse-off care product. Implements and delivery devices can employ replaceable portions (e.g., the skin interaction portions), which can be sold separately or sold together with the rinse-off care product in a kit.

Also included herein are methods for reducing malodor. For example, one method can include applying a rinse-off personal care composition comprising a malodor control polymer comprising PVam to at least a portion of the body of a user.

Another method can include a method of reducing bacteria, comprising applying a rinse-off personal care composition comprising a malodor control polymer comprising a PVam to at least a portion of the body of a user. Whether bacteria are reduced can be determined by the Hair Pluck method described herein.

A rinse-off personal care composition may comprise from about 0.01% to about 20%, by weight of the composition, of the malodor control polymer. In a further example, the composition may comprise from about 1.0% to about 20%, by weight of the composition, of the malodor control polymer. The polyvinylamine polymer may comprise a copolymer comprising about 95% mol vinyl monomer and about 5% mol vinylformamide monomer. A rinse-off personal care composition may further comprise a particulate antimicrobial agent.

While some compositional components are listed in the methods section for illustration, the rinse-off personal care compositions in the methods can contain any combination of components as described herein.

### IV. Procedure

Hair Pluck and Self-Assessed Body Odor Clinical Methods and Results

Individuals were subject to a 3-day washout period where no antiperspirant or deodorant was used and only soap and shampoo usage was permitted. Following the 3-day washout period a 10-day treatment study followed. For 10 consecutive days, the individual's underarm was limited to one treatment per day with the given tested trial product, and the individual was not permitted to use any antiperspirant or deodorant product. The dose of the tested trial product (e.g., rinse-off personal care composition) was limited to 2.5 g/underarm. Both the antimicrobial activity (Hair Plucking) and anti-odor (Self Assessments) efficacy of the compositions were determined simultaneously in the same studies.

For Assessment of Antimicrobial Activity: Replicate hairs were plucked and removed from the underarm at baseline (end of the washout period) and about 5 hours after the final (10th) treatment and placed individually into separate Soleris vials. The vials were incubated at 37° C. in a Soleris photometer and continuously monitored for 48 hours. Follicular-associated bacteria present on the hair metabolize carbohydrates in the vials generating acid by products, decreasing the culture pH, and causing a color change in a pH indicator present in the vial. The time (hours) to color change, known as the detection time, is inversely proportional to the number and/or metabolic health of follicular bacteria carried over on the hair. That is, the longer the detection time the fewer in number or less “healthy” the follicular bacteria. Longer detection times can be indicative of a strong antimicrobial effect within the hair follicle while shorter times can suggest a weak or non-existent antimicrobial effect.

For Assessment of Anti-Odor Efficacy: Subjects self-assessed their underarm odor at baseline and twice/day (am & pm) throughout the clinical trial. For their individualized odor assessment, each subject removed their shirt, turned their head towards the underarm and sniffed. An odor score was assigned for each underarm using a 0-10 rating scale where 0 indicated no detectable odor and 10 represented the highest level of odor this subject had personally experienced on themselves. Underarm odor scores were designated as “12 hour” and “24 hour”; where “12 hour” scores represent odor measures taken in the evening, or about 12 hours after product use and “24 hour” scores represent odor measures taking the following morning, or about 24 hours after product use.

### V. Examples

### A. Examples 1-2

Table 1 below illustrates two formulations for rinse-off personal care compositions. Example 1 is a placebo formulation, which is used as a comparative example for Example 2, which contains 2% PVam (Lupamin® 1595).

TABLE 1

| | % Raw Material | |
|---|---|---|
| | Example 1: Rinse- | Example 2: Rinse- |
| Ingredient | off Placebo | off with PVam |
| Distilled Water | 45.7880 | 26.7404 |
| Glycerin- USP | 2.3390 | 2.3390 |
| Polyvinyl Alcohol | 4.8000 | 4.8000 |
| Sodium Laureth-3-Sulfate | 28.5712 | 28.5712 |
| Sodium Lauryl Sulfate | 16.5518 | 16.5518 |
| Cetyl Alcohol | 0.7200 | 0.7200 |
| Coconut monoethanolamide | 1.2000 | 1.2000 |
| Kathon | 0.0300 | 0.0300 |
| PVam(Lupamin ® 1595) | — | 19.0476 |
| Total % | 100.0000 | 100.0000 |

A rinse-off personal care composition for Example 1 was formed by combining distilled water with glycerin, followed by slowly adding polyvinyl alcohol while stirring and heating to 85° C. Sodium laureth-3-sulfate, sodium lauryl sulfate, cetyl alcohol, coconut monoethanolamide, and Kathon were added to the mixture, which was stirred until uniform and then cooled to room temperature. Distilled water was then added (by weight) to replace any water that had been lost during heating. The pH of the final mixture was adjusted to between 5.0-7.0. For Example 2, Lupamin® 1595 was added to the Example 1 mixture while stifling to a desired final concentration.

### B. Examples 3-4

Table 2 illustrates two formulations for rinse-off personal care compositions. Example 3 is a rinse-off personal care composition including ZPT and ZC, where Example 3 is used as a comparative example for Example 4, which further contains 1% PVam (Lupamin® 1595).

FIGS. 1-3 show a comparison between Example 1 (“Placebo”) and Example 2 (“Lupamin 1595”). For example, FIGS. 1-2 display results for a self-assessed body odor study, comparing each of Examples 1-2 at 12 and 24 hours post-product application, respectively. As described above, for self-assessed body odor, subjects are asked to sniff under each underarm twice daily at 12 and 24 hours post-product application and record the level of odor on a scale from 0-10. As can be seen in FIGS. 1-2, Example 2, the product containing PVam (Lupamin® 1595) had superior anti-odor efficacy at both 12 & 24 hours following application versus Example 1, a placebo formulation that did not contain PVam (Lupamin® 1595). With respect to antimicrobial activity, Example 2 did not appear to exhibit noticeable improvement in antimicrobial activity when compared to baseline and Example 1, as can be seen in FIG. 3.

TABLE 2

| | % Raw Material | |
|---|---|---|
| | | Example 4: |
| | Example 3: | Rinse-off |
| | Rinse-off | with ZPT/ZC and |
| Ingredient | with ZPT/ZC | PVam |
| Distilled Water | 59.6355 | 50.1155 |
| Thixcin ® Base (see below) | 38.2600 | 38.2600 |
| Sodium Chloride | 1.4000 | 1.4000 |
| Zinc Carbonate | 0.5000 | 0.5000 |
| ZPT FPS, 48.9% active | 0.2045 | 0.2045 |
| PVam (Lupamin ® 1595) | — | 9.5200 |
| Total % | 100.0000 | 100.0000 |
| Thixcin ® Base: | | |
| Sodium Laureth-3-Sulfate (28% Active) | 19.8471 | 19.8471 |
| Sodium Lauryl Sulfate (29% Active) | 12.2367 | 12.2367 |
| Cocoamidopropyl Betaine High pH, | 4.7289 | 4.7289 |
| 30% active | | |
| Hydrochloric Acid, 6N Volumetric | 0.7576 | 0.7576 |
| Solution | | |
| Thixcin ® R | 0.3549 | 0.3549 |
| Sodium Benzoate, NF | 0.2957 | 0.2957 |
| Kathon CG, 1.5% active | 0.0391 | 0.0391 |
| Total % | 38.2600 | 38.2600 |

A rinse-off personal care composition for Example 3 was prepared by forming a Thixcin® base mix by combining sodium laureth-3-sulfate, sodium lauryl sulfate, cocoamidopropyl betaine (high pH), 6N hydrochloric acid, trihydroxystearin (Thixcin® R), sodium benzoate, and Kathon using high-shear mixing and heating until about 90° C. A separate solution was prepared by mixing water and zinc carbonate at a high speed (about 3,000-8,000 rpm) for 3-5 min. With the stifling set to about 300-500 rpm, the Thixcin® base mix and sodium chloride were added to the water/zinc carbonate slurry until a uniform solution was observed. Zinc pyrithione and water were combined in a separate solution and this resulting slurry was added to the Thixcin®/zinc carbonate mixture and then stirred for a minimum of 20 min. For Example 4, a solution of Lupamin® 1595 and water was added to the formulation for Example 3 with constant stifling until a uniform solution was achieved at a desired level of Lupamin® 1595.

FIGS. 4-6 show a comparison between Example 3 (“ZPT/ZC”) and Example 4 (“ZPT/ZC+Lupamin 1595”). FIG. 4 shows results for a comparison of antimicrobial activity for each of Examples 3-4. As can be seen in FIG. 4, Example 4, a rinse-off personal care composition including PVam (Lupamin® 1595) and a combination of ZPT and ZC, can exert a stronger antimicrobial effect against key odor-causing bacteria than that exerted by Example 3, the control composition without PVam. With respect to self-assessed odor control, Example 4 can provide superior odor control when compared with Example 3. As shown in FIG. 5, at 12 hours following product application, Example 4 provided a slight advantage over Example 3 in self-assessed odor control, but at 24 hours following application (FIG. 6), a more noticeable self-assessed odor control was exhibited by Example 4.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The products and methods/processes of the present disclosure can comprise, consist of, and consist essentially of the essential elements and limitations of the invention described herein, as well as any of the additional or optional ingredients, components, steps, or limitations described herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of reducing underarm malodor, comprising:
applying a rinse-off personal care composition to an underarm of a user, wherein the composition comprises an antimicrobial polyvinylamine copolymer comprising about 95% mol vinyl monomer and about 5% mol vinylformamide monomer.

2. The method of claim 1, wherein the rinse-off personal care composition comprises from about 0.01% to about 20%, by weight of the composition, of the malodor control polymer.

3. The method of claim 1, wherein the rinse-off personal care composition comprises a body wash or a bar soap.

4. The method of claim 1, further comprising controlling odor for at least 12 hours after application of the rinse-off personal care composition.

5. The method of claim 1, further comprising controlling odor for at least 24 hours after application of the rinse-off personal care composition.

6. The method of claim 1, wherein the composition further comprises zinc pyrithione.

7. The method of claim 6, wherein the composition further comprises zinc carbonate.

8. A method of reducing bacteria on skin, comprising:
applying a rinse-off personal care composition to at least a portion of the skin of a user, wherein the composition comprises a malodor control polymer comprising a polyvinylamine copolymer comprising a vinyl monomer and a vinyl formamide monomer.

9. The method of claim 8, wherein the rinse-off personal care composition comprises from about 0.01% to about 20%, by weight of the composition, of the malodor control polymer.

10. The method of claim 9, wherein the polyvinylamine copolymer comprises about 95% mol vinyl monomer and about 5% mol vinylformamide monomer.

11. The method of claim 8, wherein the composition further comprises a particulate antimicrobial agent.

12. The method of claim 11, wherein the composition further comprises a zinc salt.

13. The method of claim 12, wherein the particulate antimicrobial agent comprises zinc pyrithione and is present in the composition in an amount of about 0.01% to about 4%, by weight of the composition.

14. The method of claim 13, wherein the zinc salt comprises zinc carbonate and is present in the composition in an amount of about 0.10% to about 10%, by weight of the composition.

15. The method of claim 13, wherein at least a portion of the zinc pyrithione is deposited in an underarm hair follicle.

16. The method of claim 15, further comprising controlling odor for at least 12 hours after application of the rinse-off personal care composition.

17. The method of claim 15, further comprising controlling odor for at least 24 hours after application of the rinse-off personal care composition.

18. The method of claim 8, wherein the bacteria comprise S. epidermidis, C. mucifaciens, or a combination thereof.

19. The method of claim 8, wherein the rinse-off personal care composition comprises a body wash or a bar soap.
